(19) United States
(12) Patent Application Publication
(10) Pub. No.: US 2008/0317835 A1
(43) Pub. Date: Dec. 25, 2008

(54) Personalized Nutrient Deficiency Assay Methods

(75) Inventors: Nazli Azimi, San Juan Capistrano, CA (US); Mohammad Saeed Kharazmi, Corona Del Mar, CA (US)

Correspondence Address: NAZLI AZIMI, 27462 PASEO BOVEDA, SAN JUAN CAPISTRANO, CA, 92675, US

(73) Assignee: NUGENE INC.

(21) Appl. No.: 11/765,977

(22) Filed: Jun. 20, 2007

(51) Int. Cl. A61K 9/127; A61K 31/07; A61K 31/122; A61K 31/164; A61K 31/19; A61K 31/355; A61K 31/51; A61K 31/525; A61K 31/59; A61K 31/70; A61K 31/714; A61K 33/04; A61K 33/06; A61K 33/30; A61K 38/02; A61K 38/16; A61K 38/18; A61K 38/19; G01N 33/84; G01N 33/82; G01N 33/68; G01N 33/00; C12Q 1/02; A61P 43/00; A61K 38/20; A61K 38/28

(52) U.S. Cl. 424/450; 424/641; 424/682; 424/702; 424/85.2; 435/29; 436/111; 436/128; 436/131; 436/79; 436/81; 436/84; 436/86; 436/89; 436/91; 436/93; 436/94; 436/98; 514/12; 514/167; 514/2; 514/23; 514/249; 514/251; 514/276; 514/3; 514/440; 514/458; 514/52; 514/558; 514/562; 514/667; 514/699; 514/725; 514/789

(57) ABSTRACT

Assays are preformed to determine nutrient deficiency levels in an individual to address the deficiencies and improve health and longevity. Nutraceutical and cosmeceutical products containing nanoencapsulated active ingredients are provided, in addition to dietary, exercise, sleep, and other lifestyle recommendations.

### BACKGROUND

The present disclosure relates to providing a personalized health regimen for an individual based upon various assays.

### SUMMARY

Assays are preformed to determine nutrient deficiency levels in an individual to address the deficiencies and improve health and longevity. Nutraceutical and cosmeceutical products containing nanoencapsulated active ingredients are provided, in addition to dietary, exercise, sleep, and other lifestyle recommendations.

According to a feature of the present disclosure, a method is disclosed comprising obtaining data from at least one nutrient deficiency assay to determine a set of nutrient deficiencies in an individual, obtaining data from at least one genetic variation assay to determine at least one gene of the individual contributing to a nutrient deficiency; and providing at least one health recommendation to the individual. The health recommendation is based upon the nutrient deficiency assay and the genetic variation assay.

According to a feature of the present disclosure, a method is disclosed comprising determining a set of nutrient deficiencies in an individual with a blood assay and a genetic assay, correlating a result set of the genetic assay to a database of known deficiencies for observed genetic data, and providing to the individual a composition having at least one active ingredient. The at least one active ingredient addresses at least one observed nutrient deficiency.

### DRAWINGS

The above-mentioned features and objects of the present disclosure will become more apparent with reference to the following description taken in conjunction with the accompanying drawings wherein like reference numerals denote like elements and in which:

FIG. 1 is a block diagram of the methods of the present disclosure.

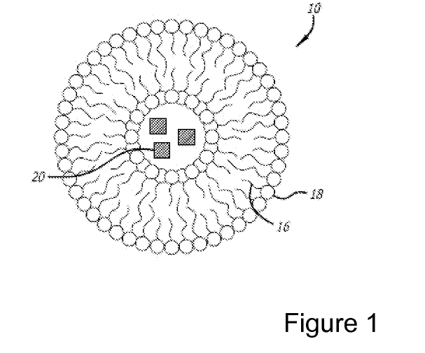

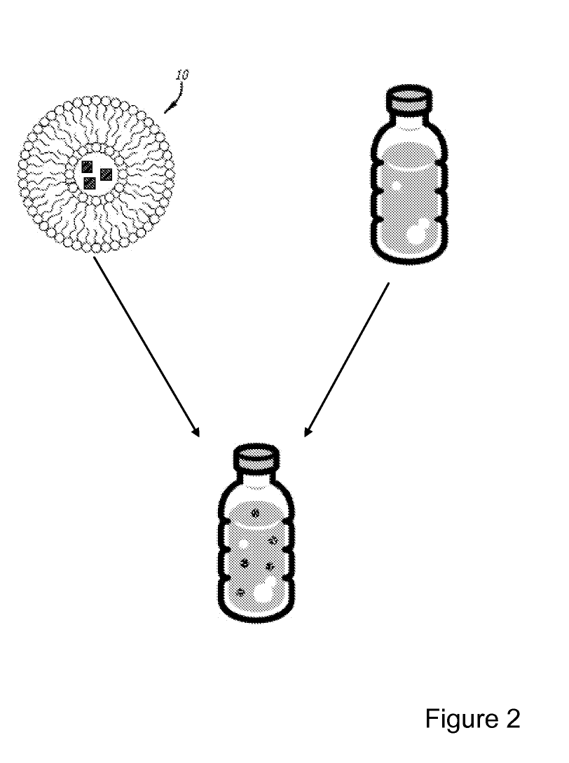

### DETAILED DESCRIPTION

In the following detailed description of embodiments of the invention, reference is made to the accompanying drawings in which like references indicate similar elements, and in which is shown by way of illustration specific embodiments in which the invention may be practiced. These embodiments are described in sufficient detail to enable those skilled in the art to practice the invention, and it is to be understood that other embodiments may be utilized and that logical, mechanical, biological, electrical, functional, and other changes may be made without departing from the scope of the present invention. The following detailed description is, therefore, not to be taken in a limiting sense, and the scope of the present invention is defined only by the appended claims. As used in the present disclosure, the term “or” shall he understood to be defined as a logical disjunction and shall not indicate an exclusive disjunction unless expressly indicated as such or notated as “xor. ”

As used in the present disclosure, the term “active ingredient” shall be defined as a component of a nutraceutical that interacts with or effects changes in tissues, cells, biological pathways, or native molecules.

As used in the present disclosure, the term “cosmeceutical” shall be defined as a substance that is applied topically to an organism. Cosmeceuticals include cosmetics with pharmaceutical properties and pharmaceutics that are applied topically.

As used in the present disclosure, the term “nutraceutical” shall be defined as a ingested substance having at least one active ingredient designed to improve the health of an organism.

According to embodiments, the present disclosure discloses a personalized health regimen for improving, preserving, or restoring an individual's health. The health regimen includes personalized suggestions and recommendations toward an individual's diet and exercise, as well as personalized nutraceuticals and cosmeceuticals formulated with active ingredients based on the nutrient deficiencies of the individual. A series of assays are conducted on the individual to provide information used for establishing the recommendations and formulations. The tests include assays for assessing nutrient levels, assays for evaluating genetic variations that impact the ability of the individual's cells to absorb nutrients, and may also include other tissue, blood, serum, skin, and hormonal tests. Based on the results of the assays, recommendations, nutraceuticals, or cosmeceuticals may be specifically developed or formulated for the individual, according to embodiments.

According to embodiments, a method is disclosed for providing a health regimen for an individual. The method comprises; (1) conducting at least one nutrient deficiency assay targeting at least one nutrient to produce a nutrient deficiency assay result; (2) conducting at least one genetic variation assay targeting at least one gene of the individual to produce a genetic variation assay result; and (3) providing at least one health recommendation to the individual based upon at least one of the nutrient deficiency assay result and genetic variation assay result.

According to embodiments, the health recommendation is selected from a diet recommendation, exercise recommendation, sleep recommendation, and lifestyle recommendation. The diet recommendation includes suggestions on specific foods to eat or to abstain from, food portions, calorie intake, fat, protein, and carbohydrate intake, and personally designed meal courses according to embodiments. Exercise recommendations include suitable exercises for a specific health condition and exercises focusing on specific muscle groups, according to embodiments. Sleep recommendations suggest how to improve sleep patterns and ensure the individual gets sufficient sleep. Lifestyle recommendations include suggestions regarding changes in the individual's current lifestyle, according to embodiments.

An important step in preserving or restoring the health of an individual is the identification and correction of nutrient deficiencies. Diseases and conditions that may be treated or prevented due to their linkage to nutrient imbalances include arthritis, Parkinson's, alcoholism/substance abuse, behavioral disorders, cancer, cardiovascular diseases, chronic fatigue, macular degeneration, diabetes, immune disorders, multiple sclerosis, stroke, and osteoporosis. Importantly, for people seeking to improve their outward appearance, nutrient deficiencies are reflected in the skin, eyes, hair and other outward indicators in a person's body.

According to embodiments, tissue, blood, and serum tests measuring quantities of individual nutrients are used to determine an individual's nutrient deficiencies. Diet and nutrient uptake is one of the many factors that influence the nutrient status of an individual. Insufficient intake or uptake of specific nutrients generally results in a deficiency of that nutrient. According to embodiments, vitamins B1, B2, B3, B6, B5, B12, D, A, and E, Folic acid, Biotin, Serine, Glutamine, Asparagine, Choline, Inositol, Carnitine, Calcium, Zinc, Magnesium, Selenium, Alpha Lipoic acid are commonly tested in blood testing experiments.

In addition, there are many other factors beyond diet that determine adequate nutrition. Because individuals are biochemically unique, nutrient deficiencies vary and do not necessarily correlate directly with nutrient intake, even among those with similar health conditions. These factors include biochemical individuality, genetic predisposition, absorption and metabolism, age, disease conditions, and medications. Assays providing information regarding an individual's nutrient status in correlation with such factors may therefore also be included as a nutrient deficiency assay.

According to embodiments, one type of nutrient deficiency assay provides information regarding nutrient deficiencies within an individual. These functional deficiencies result in defects in the biochemical pathways that depend upon the optimal function of the nutrients. A deficient or defective pathway may operate at a sub-optimal level for many months or even years before a clinical symptom may become apparent, if they become apparent at all. A functional deficiency includes anything that may reduce the concentration or the efficacy of a nutrient as compared with a normal range within a population. With a deficiency, a nutrient may be present, but it may not be properly activated, localized, or have sufficient cofactors to function at a normal level of activity. Functional deficiencies include inefficiencies or deficiencies in intracellular activation, storage concentration or activity of cofactors, and tissues with increased metabolic needs. Examples are inefficient absorption by the gastrointestinal tract, deficient transport to the appropriate tissue, impeded transport through the cell membrane, presence of intracellular inhibitors, and flaws in the biochemical pathways for the uptake of nutrients.

According to embodiments, the nutrient deficiency assay is an assay measuring levels of accumulation of the nutrient in lymphocytes of the individual. Generally, an intracellular function assay is used and comprises the steps of collecting lymphocyte cells, isolating the cells from other whole blood components, and maintaining the cells in culture during the assay. The lymphocytes collected have a 4- to 6-month lifespan in which nutrients are accumulated. The resting lymphocytes are stimulated to undergo cell division and grow in culture. The degree to which the lymphocytes grow having various nutrients available is directly related to the nutrient levels accumulated in the lymphocytes. For example, if the lymphocytes are able to grow in an environment deficient in vitamin C, then the lymphocyte has efficiently uptakes and stored vitamin C prior to harvest. On the other hand, if the lymphocyte is unable to grow in the absence of vitamin C, a deficiency is indicated. From the lymphocyte degree of growth, a functional intracellular analysis of a broad range of nutrient deficiencies may be obtained.

According to embodiments, the nutrient targeted in the nutrient deficiency assay is selected from vitamins, minerals, amino acids, antioxidants, and metabolites. According to embodiments, as shown in Table 1, the nutrient is a vitamin such as vitamin A, B1, B2, B3, B6, B12, D, E, biotin, folate, and panothenate; minerals such as calcium, magnesium, selenium, and zinc; an amino acid such as asparagine, carnitine, glutamine, and serine; an antioxidant selected from coenzyme Q10, glutathione, and cysteine; or a metabolite such as lipoic acid, oleic acid, choline, inositol, fructose, glucose, and insulin. Other vitamins, minerals, antioxidants, and nutrients may also be targeted in the nutrient deficiency assay.

TABLE 1

| Nutrient Type | Specific Nutrient |
|---|---|
| Vitamin | vitamin A, B1, B2, B3, B6, B12, D, E, biotin, |
| | folate, panothenate |
| Mineral | calcium, magnesium, selenium, zinc |
| Amino Acid | asparagine, carnitine, glutamine, serine |
| Antioxidant | coenzyme Q10, glutathione, cysteine |
| Metabolite | lipoic acid, oleic acid, choline, inositol, fructose, |
| | glucose, insulin |

According to embodiments, genetic testing is used to provide information regarding an individual's health based on the individual's genetic conditions. An individual's inherited health risks and potential health problems may be assessed through the genetic testing. More importantly, correlations maybe drawn to nutrient deficiencies based on sets of previously observed genetic mutations and nutrient deficiencies. Thus, genetic assays may lead to information regarding the cause of nutrient deficiencies or nutrient deficiencies that are unobserved in other assay methods. This allows for the development of a suitable diet, lifestyle, and supplement regimen that matches the unique nutrient deficiencies of each individual.

According to embodiments, the genetic variation assay is an assay searching for single nucleotide polymorphisms (SNPs) in the gene. A SNP is a natural variation or change in a single nucleotide basepair of a gene, which may affect the way an individual's body responds to certain stimuli such as damage, infection, or even nutrient intake. Cells are collected and select segments of the genetic code are tested for SNPs. The cells are preferably collected from a mouth rinse procedure, but can also be collected from a blood sample or other methods. The genetic test evaluates SNPs that may lead to deficiencies when influenced by other external and environmental factors. Based on the genetic test results, a customized treatment plan may be developed.

Functional assessment tests may also be conducted to monitor if an individual's deficiencies are related to breakdown in the uptake pathways or if cofactors or related biomolecule deficiencies are actually the root cause of an observed deficiency. For example, a calcium deficiency maybe observed in an individual. However, the root cause of the deficiency may breakdown in the conversion of vitamin D to 1,25 dihydroxyvitamin D, which is necessary in the production of TRPV6—a protein necessary for calcium absorption in the intestine. Functional assessment tests may be used to determine if biochemical pathways are functioning inefficiently and target growth factors and other active ingredients that induce correct function of the pathways. Thus, deficiencies may be identified not only from the perspective of intake of nutrients, but also efficiency of uptake of available nutrients.

According to embodiments, the gene assayed is at least one of TNF-α, IL-6, IL-1β, SOD1, SOD2, CAT, EPHX1, GPX1, MnSOD2, p53, IGF, TGFβ1, MMP, EGF, ELA2, VEGF, bFGF, APOE, MTHFR, and ACE. Genes evaluated include but are not limited to the genes shown in the following Table 2.

TABLE 2

| Gene | Full Name | SNP Location |
|---|---|---|
| TNF~α | tumor necrosis factor α | −308G/A |
| IL~6 | interleukin 6 | −174C/G |
| IL~1β | interleukin 1β | −31C/T |
| SOD1 and SOD2 | superoxide dismuatse 1 and 2 | 47C/T |
| CAT | Catalase | −262G/A |
| EPHX1 | microsomal epoxide | 113T/C |
| | hydrolase 1 | |
| GPX1 | Glutathione peroxidase | 593C/T |
| MnSOD2 | Manganese superoxide | 399T/C |
| | dismuatse | |
| P53 | Tumor suppressor | 72 codon Proline to |
| | | Arginine |
| IGF | Insulin Like growth factor | 192 bp allele of IGF1 |
| | | promoter |
| TGFβ1 | transforming growth factor β1 | +29T/C |
| MMP | Matrix metalloproteinase | −160G/GG |
| EGF | Epidermal growth Factor | 61G/A |
| ELA2 | Neutrophil elastase | ~903T/G and - |
| | | 714G/A |
| VEGF | Vascular Endothelial growth | 405G/C |
| | factor | |
| bFGF | Basic Fibroblast Growth | ~553T/A |
| | Factor | |
| APOE | apolipoprotein E | 388C/T |
| MTHFR | methylenetrahydrofolate | 776C/G |
| | reductase | |
| ACE | angiotensin I-converting | Insertion/deletion at |
| | enzyme | intron 16 |

According to embodiments, the method for providing a health regimen comprises formulating at least one nutraceutical or cosmeceutical based upon at least one of the nutrient deficiency assay result and genetic variation assay result. Nutraceuticals include vitamins, minerals, growth factors, antioxidants, metabolites, etc., to address specific nutrient deficiencies of the individual if a nutrient is inefficiently absorbed, higher doses of the nutrient may correct the deficiency in a “brute force” manner, or the machinery for uptake of the nutrient may be repaired to increase uptake, according to embodiments.

According to embodiments, a health regimen is provided for an individual. The health regimen comprises: (1) at least one nutrient deficiency assay targeting at least one nutrient of the individual, wherein the nutrient deficiency assay produces a nutrient deficiency assay result; (2) at least one genetic variation assay targeting at least one gene of the individual, wherein the genetic variation assay produces a genetic variation assay result; and (3) at least one health recommendation to the individual, wherein the health recommendation is based upon at least one of the nutrient deficiency assay result and genetic variation assay result.

The health recommendation comprises, according to embodiments, dietary suggestions for increasing deficient nutrients. For example, individuals who are deficient in folic acid will be instructed to eat at least one helping of leafy green vegetables each day. Similarly, exercise, sleep, and other lifestyle-type regimens may be recommended. For example, a person who, in addition to nutrient deficiencies, is tested to have elevated catecholamine and glucocorticoid hormones associated with stress will receive a recommendation to exercise for 30 minutes a day, sleep 7-8 hours per night, and reduce the amount of time spent at work on the weekends. Thus, in addition to providing nutraceuticals or cosmeceuticals, other natural approaches are applicable to improve health and appearance, as well as increase longevity generally.

According to embodiments, the health recommendation further comprises at least one nutraceutical or cosmeceutical having active ingredients, wherein formulation of the nutraceutical or cosmeceutical is based upon at least one of the nutrient deficiency assay result and genetic variation assay result.

According to embodiments, further assays may be performed to increase the accuracy or scope of the nutrient deficiency assay and the genetic variation assays. These assays may comprise hormone assays, which detect hormonal imbalances that may be addressed, at least in part, with metabolites, growth factors, or other biomolecules, according to embodiments. According to other embodiments, a skin test may also be used to determine deficiencies by observing the nutrient levels in skin cells.

### EXAMPLE 1

According to embodiments, an IFA is used to measure levels of accumulation of a nutrient in lymphocytes of the individual. The assay provides information regarding nutrient functional deficiencies within an individual. For example, the IFA is conducted with steps as disclosed herein. Blood is first collected from an individual. Fasting is not required for this process. The collection of blood is accomplished preferably through venipuncture, though other methods may be possible, for example metabolite testing from the urine. The blood is sent to a laboratory where lymphocyte cells are isolated and grown in a series of culture media containing optimal amounts of specific micronutrients. Utilizing a lymphocyte-specific mitogen, for example PHA and PMA, r

the lymphocytes are stimulated to undergo cell division and grow in the control media. The specific micronutrients are removed from the media separately, forcing the cells to use their own internal mechanisms (reserves or metabolic processes) to compensate for the removed micronutrient. If the cells are able to grow optimally following the removal of a specific micronutrient, the cells are functioning adequately. If the cells are not able to grow optimally following the removal of a specific micronutrient, then a deficiency related to the removed micronutrient is indicated. As an example, when B12 is removed from the media and cell growth is not sufficient, a functional intracellular deficiency of B12 is indicated.

While the apparatus and method have been described in terms of what are presently considered to be practical embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising;
obtaining data from at least one nutrient deficiency assay to determine a set of nutrient deficiencies in an individual;
obtaining data from at least one genetic variation assay to determine at least one gene of the individual contributing to a nutrient deficiency; and
providing at least one health recommendation to the individual;
wherein the health recommendation is based upon the nutrient deficiency assay and the genetic variation assay.

2. The method of claim 1, further comprising formulating at least one nutraceutical based upon at least one of the nutrient deficiency assay and genetic variation assay.

3. The method of claim 1 wherein the nutrient deficiency assay is an assay measuring levels of at least one nutrient in lymphocytes.

4. The method of claim 1, wherein the nutrient is selected from vitamins, minerals, amino acids, antioxidants, and metabolites.

5. The method of claim 4, wherein the vitamin is selected from vitamin A, B1, B2, B3, B6, B12, D, E, biotin, folate, and panothenate.

6. The method of claim 4, wherein the mineral is selected from calcium, magnesium, selenium, and zinc.

7. The method of claim 4, wherein the amino acid is selected from asparagine, carnitine, glutamine, and serine.

8. The method of claim 4, wherein the antioxidant is selected from coenzyme Q10, glutathione, and cysteine.

9. The method of claim 4, wherein the metabolite is selected from lipoic acid, oleic acid, choline, inositol, fructose, glucose, and insulin.

10. The method of claim 1, wherein the genetic variation assay is an assay for locating at least one single nucleotide polymorphism in a set of assayed genes.

11. The method of claim 1, wherein the set of genes comprises TNF-α, IL-6, IL-1β, SOD1, SOD2, CAT, EPHX1, GPX1, MnSOD2, p53, IGF, TGFβ1, MMP, EGF, ELA2, VEGF, bFGF, APOE, MTHFR, and ACE.

12. The method of claim 1, wherein the health recommendation comprises administration of at least one active ingredient to address observed deficiencies.

13. The method of claim 12, wherein the health recommendation further comprises at least one of a diet recommendation, an exercise recommendation, a sleep recommendation, and a lifestyle recommendation.

14. A method comprising:
determining a set of nutrient deficiencies in an individual with a blood assay and a genetic assay;
correlating a result set of the genetic assay to a database of known deficiencies for observed genetic data; and
providing to the individual a composition having at least one active ingredient;
wherein the at least one active ingredient addresses at least one observed nutrient deficiency.

15. The method of claim 14, wherein the composition is a nutraceutical.

16. The method of claim 14, wherein the composition is a cosmeceutical.

17. The method of claim 14, wherein the at least one active ingredient comprises a vitamin, a mineral, an amino acid, an antioxidant, a growth factor, peptide fragments mimicking the activity of growth factors, or a biomolecule.

18. The method of claim 14, wherein:
the vitamin comprises at least one of vitamin A, B1, B2, B3, B6, B12, D, E, biotin, folate, and panothenate;
the mineral comprise at least one of calcium, magnesium, selenium, and zinc;
the antioxidant comprise at least one of coenzyme Q10, glutathione, and cysteine;
the metabolite comprise at least one of lipoic acid, oleic acid, choline, inositol, fructose, glucose, and insulin; and
the growth factors comprise at least one of TNF-α, IL-6, IL-1β, SOD1, SOD2, CAT, EPHX1, GPX1, MnSOD2, p53, IGF, TGFβ1, MMP, EGF, ELA2, VEGF, bFGF, APOE, MTHFR, and ACE.

19. The method of claim 14, wherein the blood assay comprises a determination of the level of at least one nutrient in lymphocytes and the genetic assay determines the presence of short nucleotide polymorphisms in a set of genes.

20. The method of claim 14, wherein at least one active ingredient is encapsulated in a nanosome.
